(19) United States
(12) Patent Application Publication
(10) Pub. No.: US 2011/0038922 A1
(43) Pub. Date: Feb. 17, 2011

(54) COMPOUNDS FOR TREATING OR PREVENTING AMINE OXIDASE RELATED DISEASES OR DISORDERS

(75) Inventors: Sirpa Jalkanen, Piispanristi (FI); Markku Jalkanen, Piispanristi (FI); Marko Salmi, Turku (FI)

Correspondence Address: ROTHWELL, FIGG, ERNST & MANBECK, P.C., 1425 K STREET, N.W., SUITE 800, WASHINGTON, DC, 20005, US

(73) Assignee: FARON PHARMACEUTICALS OY (A FINNISH COMPANY), Turku (FI)

(21) Appl. No.: 11/917,638

(22) PCT Filed: Jun. 12, 2006
(86) PCT No.: PCT/FI2006/000188

§ 371 Date: Sep. 17, 2008

(30) Foreign Application Priority Data
Jun. 16, 2005 (FI) 20050640

(51) Int. Cl. A61K 9/127; A61K 31/7105; C12N 15/63

(52) U.S. Cl. 424/450; 435/320.1

(57) ABSTRACT

This invention relates to small interfering RNAs (siRNAs) that down regulates the expression of vascular adhesion protein 1 (VAP-1) for prevention or treatment of a disease or disorder that benefits from the inhibition or down regulation of VAP-1. Pharmaceutical compositions comprising said siRNAs in combination with pharmaceutically acceptable carriers are also included. Furthermore, the invention concerns expression vectors comprising nucleic acids encoding the siRNA duplexes or the antisense strands of said duplexes in a manner which allows expression of said siRNA duplexes or antisense strands within a mammalian cell. Pharmaceutical compositions comprising said expression vectors in combination with pharmaceutically acceptable carriers are also included.

### FIELD OF THE INVENTION

This invention relates to the use of a small interfering RNA (siRNA) duplexes for down regulation of the expression of vascular adhesion protein 1 (VAP-1) and for treating or preventing diseases or disorders benefiting from such down regulation. Further, the invention concerns pharmaceutical compositions of said siRNA:s. The invention concerns also expression vectors encoding said siRNA duplexes or the antisense strands thereof as well as the use of such vectors and their pharmaceutical compositions.

### BACKGROUND OF THE INVENTION

The publications and other materials used herein to illuminate the background of the invention, and in particular, cases to provide additional details respecting the practice, are incorporated by reference.

VAP-1 is a human endothelial cell adhesion molecule that has several unique properties that distinguish it from the other inflammation-related adhesion molecules. It has a unique and restricted expression pattern and mediates lymphocyte binding to vascular endothelium (Salmi, M., and Jalkanen, S., Science 257:1407-1409 (1992)). Inflammation induces the upregulation of VAP-1 to the surface of vascular endothelial cells mediating leukocyte entry to skin, gut and inflamed synovium (Salmi, M., and Jalkanen, S., Science 257:1407-1409 (1992); Salmi, M, et al., J. Exp. Med 178:2255-2260 (1993); Arvilommi, A., et al., Eur. J. Immunol 26:825-833 (1996); Salmi, M., et al., J. Clin. Invest. 99:2165-2172 (1997): (Salmi. M., and Jalkanen, S., J. Exp. Med. 183:569-579 (1996); J. Exp. Med 186:589-600 (1997)). One of the most interesting features of VAP-1 is a catalytic extracellular domain which contains a monoamine oxidase activity (Smith, D. J., et al., J. Exp. Med 188:17-27 (1998)).

The cloning and sequencing of the human VAP-1 cDNA revealed that it encodes a transmembrane protein with homology to a class of enzymes called the copper-containing amine oxidases (E.C. 1.4.3.6). Enzyme assays have shown that VAP-1 possesses a monoamine oxidase (MAO) activity which is present in the extracellular domain of the protein (Smith, D. J., et al., J. Exp. Med. 188:17-27 (1998)). Thus, VAP-1 is an ecto-enzyme. Analysis of the VAP-1 MAO activity showed that VAP-1 belongs to the class of membrane-bound MAO's termed semicarbazide-sensitive amine oxidases (SSAO). These are distinguished from the widely distributed mitochondrial MAO-A and B flavoproteins by amino acid sequence, cofactor, substrate specificity and sensitivity to certain inhibitors. However, certain substrates and inhibitors are common to both SSAO and MAO activities. The mammalian SSAO's can metabolize various monoamines produced endogenously or absorbed as dietary or xenobiotic substances. They act principally on primary aliphatic or aromatic monoamines such as methylamine or benzylamine (Lyles G. A., Int. J. Biochem. Cell Biol, 28:259-274 (1996)). Thus, VAP-1 located on the vascular endothelial cell surface can act on circulating primary monoamines with the following reaction pathway.

RNH₂+O₂+H₂O→RCHO+H₂O₂+NH₃

The physiological substrates of VAP-1 SSAO in man have not been clearly identified. However, methylamine is a good substrate for VAP-1 SSAO. Methylamine is a product of various human biochemical pathways for the degradation of creatinine, sarcosine and adrenaline, and is found in various mammalian tissues and in blood. It can also be derived from the diet by gut bacterial degradation of dietary precursors. The concentration of methylamine in the blood can be increased in certain physiological and pathological situations such as diabetes. Another potential physiological substrate is aminoacetone.

VAP-1 SSAO activity has been proposed to be directly involved in the pathway of leukocyte adhesion to endothelial cells by a novel mechanism involving direct interaction with an amine substrate presented on a VAP-1 ligand expressed on the surface of a leukocyte (Salmi et al. Immunity, (2001)). This publication describes the direct involvement of VAP-1 SSAO activity in the process of adhesion of leukocytes to endothelium. Moreover, deletion of VAP-1 from mouse confirms the importance of VAP-1 in vivo. VAP-1 deficient animals have decreased leukocyte infiltration at the sites of inflammation (e.g. in peritonitis) when compared to their wild-type littermate controls (Stolen et al. Immunity 22:105, 2005). Thus inhibitors of VAP-1 SSAO activity could be expected to reduce leukocyte adhesion in areas of inflammation and thereby reduce leukocyte trafficking into the inflamed region and therefore the inflammatory process itself.

In human clinical tissue samples expression of VAP-1 is induced at sites of inflammation. This increased level of VAP-1 can lead to increased production of H₂O₂ generated from the action of the VAP-1 SSAO extracellular domain on monoamines present in the blood. This generation of H₂O₂ in the localized environment of the endothelial cell could initiate other cellular events. H₂O₂ is a known signaling molecule that can upregulate other adhesion molecules and this increased adhesion molecule expression may lead to enhanced leukocyte trafficking into areas in which VAP-1 is expressed. It also may be that other products of the VAP-1 SSAO reaction could have biological effects also contributing to the inflammatory process. Thus the products of the VAP-1 SSAO activity may be involved in an escalation of the inflammatory process which could be blocked by specific SSAO inhibitors.

VAP-1 SSAO may be involved in a number of other pathological conditions associated with an increased level of circulating amine substrates of VAP-1 SSAO. The oxidative deamination of these substrates would lead to an increase in the level of toxic aldehydes and oxygen radicals in the local environment of the endothelial cell which could damage the cells leading to vascular damage. Increased levels of methylamine and aminoacetone have been reported in patients with Type I and Type II diabetes and it has been proposed that the vasculopathies such as retinopathy, neuropathy and nephropathy seen in late stage diabetes could be treated with specific inhibitors of SSAO activity.

Various strategies for inhibiting VAP-1 activity have been disclosed. For example, WO 93/25582 discloses a monoclonal antibody specifically binding to VAP-1.

Alternatively, VAP-1 can be counteracted by using small molecules as inhibitors. The patent publications WO 02/020290 and WO 03/006003 disclose certain hydrazino compounds useful as specific VAP-1 SSAO inhibitors that modulate VAP-1 activity. These compounds are described as useful for the treatment of acute and chronic inflammatory conditions or diseases as well as diseases related to carbohydrate metabolism, aberrations in adipocyte differentiation or function and smooth muscle cell function, and various vascular diseases.

Grifantini, M., et al., Farmaco, Ed. Sci. 23(3):197-203 (1968), report the synthesis of several alkyl- and acyl-derivatives of N-amino-1-ephedrine and N-amino-d-pseudoephedrine having antidepressant and monoamine oxidase inhibitory properties. Jeffrey O'Sullivan et al., Biochimica et Biophysica Acta 1647 (2003) 367-371 report the inhibition of semicarbazide-sensitive amine oxidases by certain monoaminosubstituted hexoses, namely glucosamine, galactosamine and mannosamine.

### SUMMARY OF THE INVENTION

The aim of the present invention is to provide a new approach for counteracting the influence of VAP-1 in the individual. The expression of VAP-1 is down regulated by the influence of a small interfering RNA (siRNA) directed to a selected target site of the mRNA of VAP-1. Therefore, diseases or disorders benefiting from inhibiting VAP-1 by antibodies or small molecule inhibitors can be treated or prevented by the use of this new concept.

The novelty of siRNA based inhibition of VAP-1 function is the fact that it is the only way to inhibit both adhesive and enzymatic functions of VAP-1. It has been shown that surface epitopes of VAP-1, the function of which can be inhibited with anti-VAP-1 antibodies, are important in binding of leukocytes (Koskinen et al Blood 103:3388, 2004). However, also enzymatic SSAO activity of VAP-1 is involved in leukocyte adhesion (Koskinen). Moreover, the enzymatic activity results in the production of the biologically active end-products of the SSAO reaction (see above). It is known that anti-VAP-1 antibodies do not inhibit the SSAO activity of the molecule, and the small molecular SSAO inhibitors do not down-regulate surface expression of VAP-1 or block the epitopes seen by the anti-VAP-1 antibodies (Koskinen). Thus, these treatments only inhibit or down-regulate one aspect of VAP-1 function. In contrast, siRNA treatment, which leads to down-regulation of VAP-1 expression, simultaneously reduces the availability of the adhesive surface epitopes of VAP-1 and decreases the enzymatic SSAO activity.

Thus, according to one aspect, this invention concerns the use of a small interfering RNA (siRNA) that down regulates the expression of vascular adhesion protein 1 (VAP-1), said siRNA being a duplex comprising an antisense sequence of about 21 nucleotides, said antisense being complementary to a region of the VAP-1 mRNA, and a sense sequence that is complementary to a sequence of about 19 nucleotides of said antisense, wherein the antisense sequence and the sense sequence both comprise a 3′-terminal overhang of a few, typically 2 nucleotides, and wherein the 5′-terminal of the antisense is a phosphate group (P), in the manufacture of a pharmaceutical composition for use in prevention or treatment of a disease or disorder that benefits from the inhibition or down regulation of VAP-1.

According to another aspect, the invention concerns a pharmaceutical composition comprising the novel siRNA duplex as defined above a pharmaceutically acceptable carrier.

According to a third aspect, this invention concerns an expression vector comprising nucleic acid encoding the siRNA duplex as defined above or the antisense strand of said duplex, in a manner which allows expression of said siRNA duplex or antisense strand within a mammalian cell.

According to a fourth aspect, this invention concerns a pharmaceutical composition comprising an expression vector comprising nucleic acid encoding the siRNA duplex as defined above or the antisense strand of said duplex, in a manner which allows expression of said siRNA duplex or antisense strand within a mammalian cell, and a pharmaceutically acceptable carrier.

According to a fifth aspect, the invention concerns the use of an expression vector as defined above in the manufacture of a pharmaceutical composition for use in prevention or treatment of a disease or disorder that benefits from the inhibition or down regulation of VAP-1.

### BRIEF DESCRIPTION OF THE DRAWINGS

FIG. 1 shows the target mRNA (illustrated as cDNA) sequence of mouse VAP-1 (SEQ ID NO 1). Four alternative target sites estimated to be of particular interest are indicated in bold (target site 1: nt 981-963; target site 2 nt: 1771-1753; target site 3: nt 1818-1800; target site 4: nt 2558-2540).

FIG. 2 shows four alternative siRNA-duplexes, each comprising an antisense sequence complementary to a target site of the target mRNA, and a sense sequence. The antisense strand of siRNA no 1 is complementary to target site 1 in FIG. 1, the antisense of siRNA no 2 is complementary to target site 2, antisense of siRNA no 3 is complementary to target site 3, and antisense of siRNA no 4 is complementary to target site 4 shown in FIG. 1.

FIG. 3 shows the effect of siRNA no 1 (shown in FIG. 2) on down regulating VAP-1 expression in CHO cells compared to the effect of control siRNA against GFP, green fluorescent protein (a target protein that is not expressed in human cells and therefore serves as a negative control to exclude non-specific effects of any RNAi molecule).

FIG. 4 shows the target mRNA (illustrated as cDNA) sequence of human VAP-1 (SEQ ID NO 10). Four alternative target sites estimated to be of particular interest are indicated in bold italics (target site 1: nt 1227-1245; target site 2 nt: 1557-1575; target site 3: nt 2161-2179 ; target site 4: nt 2446-2464).

FIG. 5 shows four alternative siRNA-duplexes, each comprising an antisense sequence complementary to a target site of the target mRNA, and a sense sequence. The antisense strand of siRNA no I is complementary to target site 4 in FIG. 4, the antisense of siRNA no II is complementary to target site 1, antisense of siRNA no III is complementary to target site 2, and antisense of siRNA no IV is complementary to target site 3 shown in FIG. 4.

FIG. 6 shows the effect of siRNA no III (shown in FIG. 5) on down regulating VAP-1 expression in CHO cells compared to the effect of control siRNA against GFP.

### DETAILED DESCRIPTION OF THE INVENTION

Uses and Principle of Action of siRNA:

The application of siRNA:s has become important in the development of new therapies in the last years. O Heidenreich presents an overview of pharmaceutical applications in the article “Forging therapeutics from small interfering RNAs in European Pharmaceutical Review Issue 1, 2005. The principle has particularly been suggested for the treatment of tumors and carcinomas, sarcomas, hypercholesterolemia, neuroblastoma and herpetic stromal keratitis.

The principle of siRNA is extensively presented in literature. As examples can be mentioned the US patent publications 2003/0143732, 2003/0148507, 2003/0175950, 2003/0190635, 2004/0019001, 2005/0008617 and 2005/0043266. An siRNA duplex molecule comprises an antisense region and a sense strand wherein said antisense strand comprises sequence complementary to a target region in an mRNA sequence encoding a certain protein, and the sense strand comprises sequence complementary to the said antisense strand. Thus, the siRNA duplex molecule is assembled from two nucleic acid fragments wherein one fragment comprises the antisense strand and the second fragment comprises the sense strand of said siRNA molecule. The sense strand and antisense strand can be covalently connected via a linker molecule, which can be a polynucleotide linker or a non-nucleotide linker. The length of the antisense and sense strands are typically about 19 to 21 nucleotides each. Typically, the antisense strand and the sense strand both comprise a 3′-terminal overhang of a few, typically 2 nucleotides. The 5′-terminal of the antisense is typically a phosphate group (P).

The siRNA duplexes having terminal phosphate groups (P) are easier to administrate into the cell than a single stranded antisense. In the cell, an active siRNA antisense strand is formed and it recognizes a target region of the target mRNA. This in turn leads to cleaving of the target RNA by the RISC endonuclease complex (RISC=RNA-induced silencing complex) and also in the synthesis of additional RNA by RNA dependent RNA polymerase (RdRP), which can activate DICER and result in additional siRNA duplex molecules, thereby amplifying the response.

Thus, compared to usual antisense therapy, the siRNA therapy has the following advantages: 1) administration into the cell is easier because of the duplex form, 2) smaller doses are required because additional duplex molecules are synthesized in the cell and 3) the target RNA is destructed by cleavage.

### Preferred Embodiments

Preferred siRNA Structures:

The siRNA duplex should preferable have an antisense sequence of about 21 nucleotides, typically 19-21 nucleotides. The sense sequence that is complementary should preferably be of the same length so that it is complementary to the antisense, except for the nucleotides of the sense sequence that creates the overhang, which are not necessary complementary to the antisense. The overhangs at the 3′-terminal of the antisense and sense strands contain typically 2 nucleotides.

The term “complementary” means that the nucleotide sequence can form hydrogen bonds with the target RNA sequence by Watson-Crick or other base-pair interactions. The term shall be understood to cover also sequences which are not 100% complementary. It is believed that also lower complementarity might work. However, 100% complementarity is preferred.

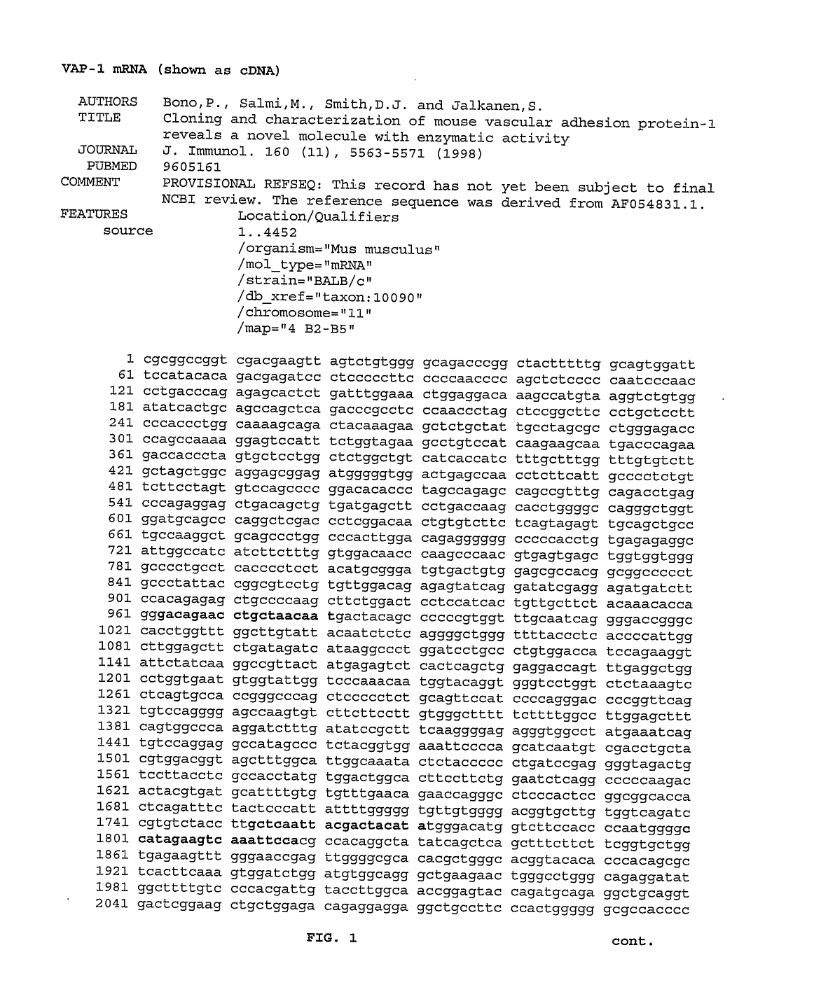

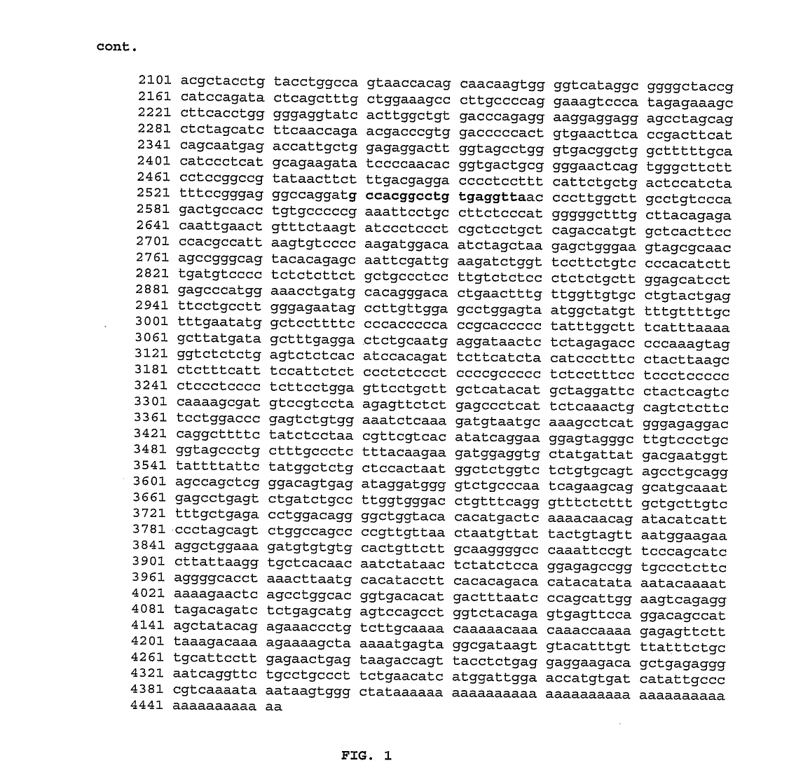

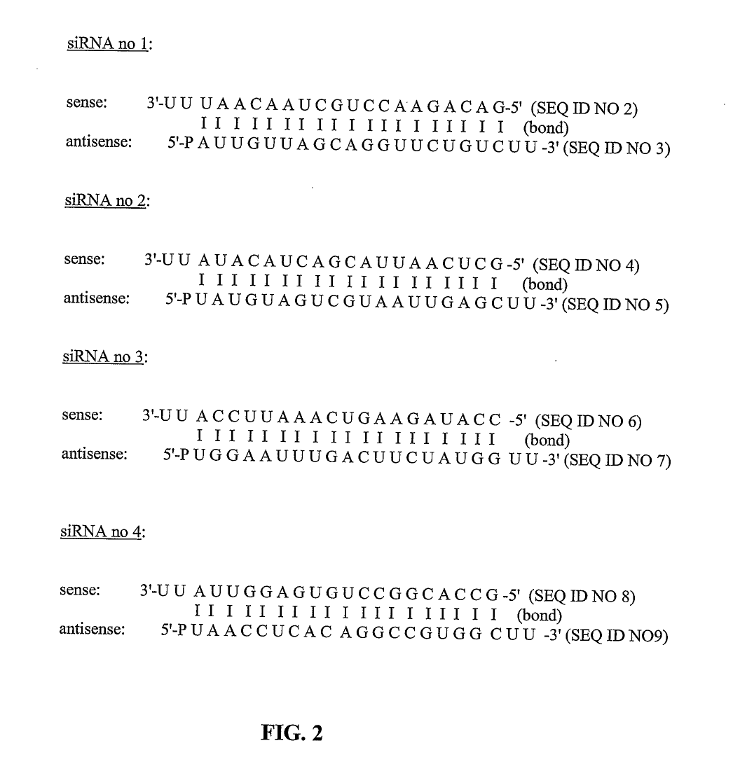

Certain preferred siRNA:s are shown in FIG. 2, which are directed to the targets marked in bold in the VAP-1 mRNA (cDNA) shown in FIG. 1.

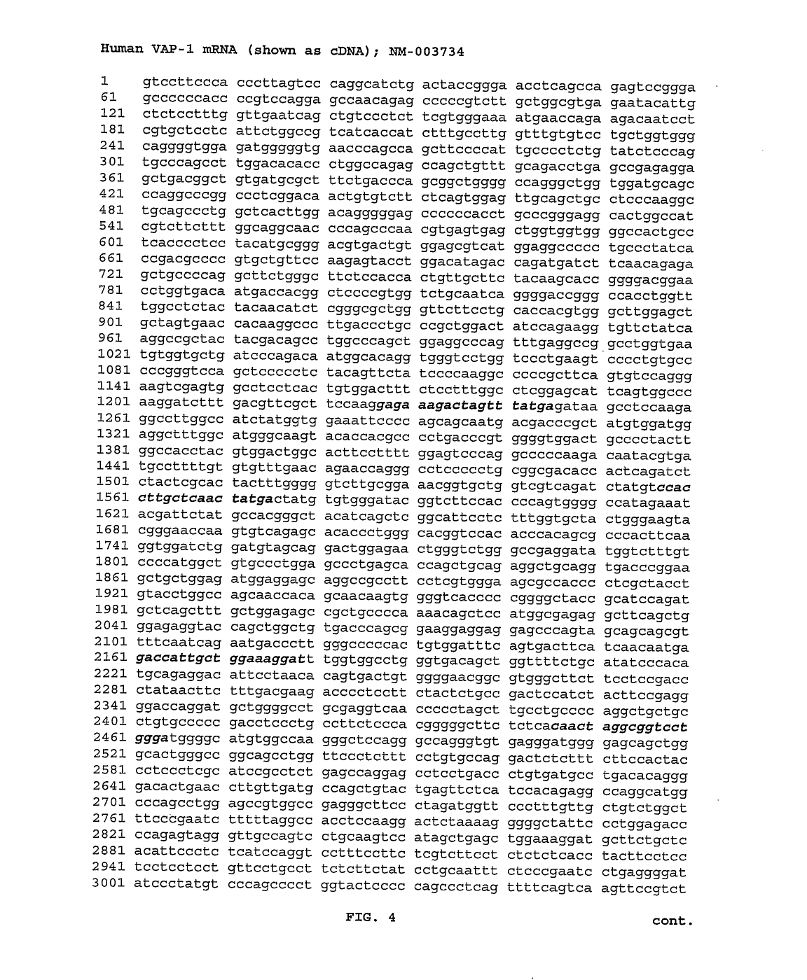

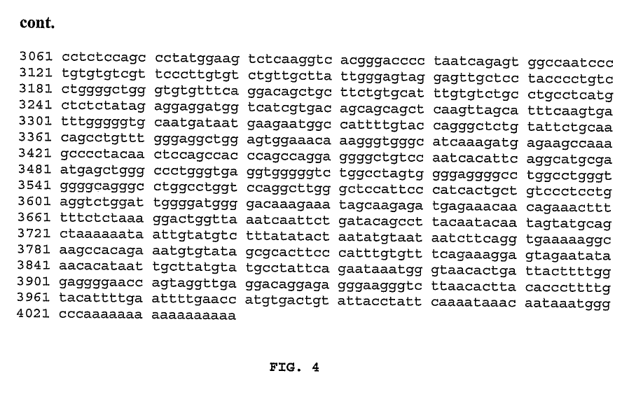

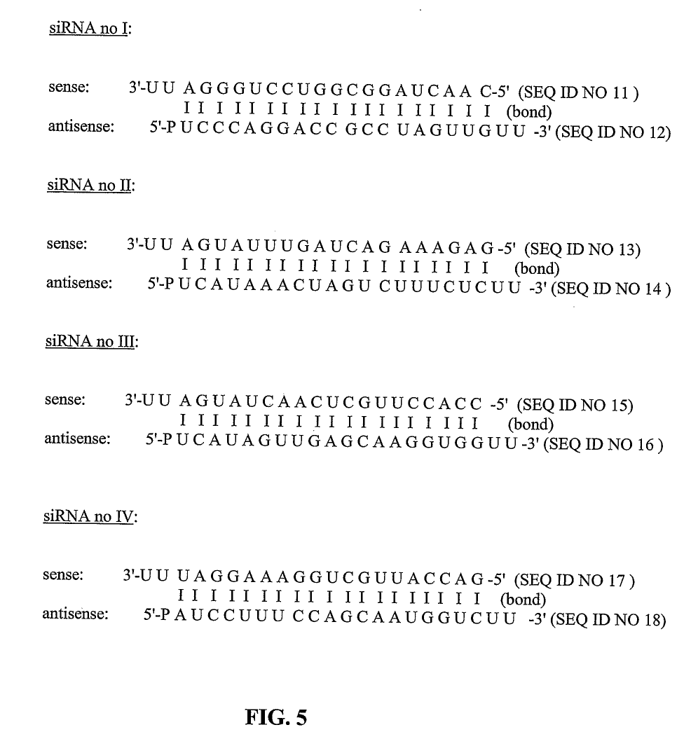

Particularly preferred are the siRNA:s shown in FIG. 5, which are directed to the targets marked in bold in the humanVAP-1 mRNA (cDNA) shown in FIG. 4.

However, also other useful target regions at the target RNA can be used. A useful target region can easily be identified by using any of the numerous academic or commercially affiliated algorithms that have been developed to assist scientists to locate utilizable siRNA sequences. As examples of such software systems can be mentioned siDirect (http://design.RNAi.jp/) (Nucleic Acids Res. 2004 Jul. 1 ;32: W124-9); TROD (T7 RNAi Oligo Designer (http://www.cellbio.unige.ch/RNAi.html; Nucleic Acids Res. 2004 Jul. 1 ;32: W121-3); DEQOR (http://cluster-1.mpi-cbg.de/Deqor/deqor.html; Nucleic Acids Res. 2004 Jul. 1 ;32: W113-20) or programs available at http://www.genscript.com; http://www.genscript.com/rnai.html#design or http://www.genscript.com/sirna_ca.html#design; Bioinformatics 2004 Jul. 22 ;20(11)1818-20. An essential criterion of the tools is to achieve siRNA:s with maximum target-specificity for mammalian RNA interference where off-target gene silencing is avoided. The usefulness of any sequence identified by such algorithms should thereafter be verified by experiments, for example by introducing it into VAP-1 positive cells, estimating the decrease in VAP-1 mRNA, the decreased VAP-1 protein expression, or the decrease in SSAO enzyme activity by using routine techniques such as quantitative reverse-transcriptase PCR, immunohistochemistry, immunocytological stainings, immunoblotting or SSAO enzyme assays. For more information, see K Huppi et al., Defining and Assaying RNAi in Mammalian Cells, Molecular Cell, Vol. 17, 1-10 Jan. 7, 2005.

Modifications:

The siRNA molecule shall, when used as a pharmaceutical, be introduced in a target cell. The delivery can be accomplished, as will be dealt with in more detail in the following section, in two principally different ways: 1) exogenous delivery of the siRNA duplex or 2) endogenous transcription of a DNA sequence encoding this siRNA duplex or the antisense strand thereof, where the DNA sequence is located in a vector.

Normal, unmodified RNA has low stability under physiological conditions because of its degradation by ribonuclease enzymes present in the living cell. If the siRNA duplex shall be administered exogenously, it is highly desirable to modify the molecule according to known methods so as to enhance its stability against chemical and enzymatic degradation.

Modifications of nucleotides, not only siRNA:s but also antisense oligonucleotides, ribozymes, etc. to be administered exogenously in vivo are extensively described in the art. Principally, any part of the nucleotide, i.e. the ribose sugar, the base and/or internucleotidic phosphodiester strands can be modified. For example, removal of the 2′-OH group from the ribose unit to give 2′-deoxyribosenucleotides results in improved stability. Prior discloses also other modifications at this group: the replacement of the ribose 2′-OH group with alkyl, alkenyl, allyl, alkoxyalkyl, halo, amino, azido or sulthydryl groups. Also other modifications at the ribose unit can be performed: locked nucleid acids (LNA) containing methylene linkages between the 2′- and 4′-positions of the ribose can be employed to create higher intrinsic stability.

Furthermore, the internucleotidic phosphodiester linkage can, for example, be modified so that one ore more oxygen is replaced by sulfur, amino, alkyl or alkoxy groups. Also the base in the nucleotides can be modified.

Preferably, the siRNA comprises modifications of one or more 2′-hydroxyl groups at ribose sugars, and/or modifications in one or more internucleotidic phosphodiester linkages, and/or one or more locked nucleic acid (LNA) modification between the 2′- and 4′-position of the ribose sugars.

Particularly preferable modifications are, for example, replacement of one or more of the 2′-OH groups by 2′-deoxy, 2′-O-methyl, 2′-halo, e.g. fluoro or 2′-methoxyethyl. Especially preferred are siRNA:s where some of the internucleotide phoshodiester linkages also are modified, e.g. replaced by phosphorothioate linkages.

It should be stressed that the modifications mentioned above are only non-limiting examples. The siRNA:s according to this invention can bear any modification.

The unmodified as well as the modified siRNA molecules can be prepared according to the methods disclosed in the cited patent publications and other prior art publications.

Administration and formulations of the siRNA:

The siRNA duplex according to this invention can be administered to the individual by various methods. According to one method, the siRNA may be administered exogenously as such, or in the form of a pharmaceutical composition admixed with a suitable carrier which may be, for example, a liposome, cholesterol, lithocholic acid, lauric acid, a cationic lipid, polyethylenimine (PEI) or its conjugates with polyethylene glycol (PEG) derivatives. However, also other carriers can be used.

The siRNA can be administered systemically or locally. As suitable routes of administration can be mentioned intravenous, intramuscular, subcutaneous injection, inhalation, oral, topical, ocular, sublingual, nasal, rectal, intraperitoneal delivery and transdermal delivery systems. The composition containing the siRNA can, instead of using direct injection, also be administered by use of, for example, a catheter, infusion pump or stent.

Another method to achieve high concentrations of the siRNA in cells is to incorporate the siRNA-encoding sequence into an expression vector and to administer such a vector to the individual. In this application, the expression vector could be construed so that either the siRNA duplex or only the antisense strand thereof is expressed, e.g. in the form of short hairpin RNAs. The expression vector can be a DNA sequence, such as a DNA plasmid capable of eukaryotic expression, or a viral vector. Such a viral vector is preferably based on an adenovirus, an alphavirus, an adeno-associated virus or a retrovirus. Preferably, the vector is delivered to the patient in similar manner as the siRNA described above. The delivery of the expression vector can be systemic, such as intravenous, intramuscular or intraperitoneal administration, or local delivery to target tissue or to cells explanted from the patient, followed by reintroduction into the patient.

Since intravenous administration of siRNA preferentially targets liver vasculature (Lewis D L and Wolff J A, Methods Enzymol. 2005;392:336-50; Soutschek J et al., Nature. 2004 Nov. 11 ;432(7014):173-8; and Song E et al., Nat Med. 2003 Mar. ;9(3):347-51), diseases of liver are especially suitable targets for intervention.

Required Dose:

The required dosage of the compounds will vary with the particular disease or condition being treated, the severity of the condition, the duration of the treatment, the administration route and the specific compound being employed.

Thus, a typical daily dose is in the dosage range of about 1 mg/kg to about 20 mg/kg, preferably about 5 mg/kg body weight. When siRNA is used, the suitable administration frequence is believed to be 1 to 2 doses daily. When the RNAi is delivered by an expression vector, a single dose (or a single doses repeated at certain intervals, e.g. once in week) is believed to be enough.

Diseases or Conditions with Responsiveness to Inhibition or Down Regulation of VAP-1:

In the following, the tern “treatment” or “treating” shall be understood to include complete curing of a disease or condition, as well as amelioration or alleviation of said disease or condition.

The term “prevention” shall be understood to include complete prevention, prophylaxis, as well as lowering the individual's risk of falling ill with said disease or condition.

As examples of groups of diseases or conditions the treatment or prevention of which would benefit from inhibition or down regulation of VAP-1 can be mentioned inflammatory diseases or conditions; diseases related to carbohydrate metabolism; diseases related to aberrations in adipocyte differentiation or function or smooth muscle cell function and vascular diseases. However, the diseases or conditions are not restricted to these groups.

According to one embodiment, the inflammatory disease or condition can be a connective tissue inflammatory disease or condition, such as, but not limited to ankylosing spondylitis, Reiter's syndrome, psoriatic arthritis, osteoarthritis or degenerative joint disease, rheumatoid arthritis, Sjögren's syndrome, Bechet's syndrome, relapsing polychondritis, systemic lupus erythematosus, discoid lupus erythematosus, systemic sclerosis, eosinophilic fasciitis, polymyositis and dermatomyositis, polymyalgia rheumatica, vasculitis, temporal arteritis, polyarterisis nodosa, Wegner's granulamatosis, mixed connective tissue disease, or juvenile rheumatoid arthritis.

According to another embodiment, said inflammatory disease or condition is a gastrointestinal inflammatory disease or condition, such as, but not limited to Crohn's disease, ulcerative colitis, irritable bowel syndrome (spastic colon), fibrotic conditions of the liver, inflammation of the oral mucosa (stomatitis), or recurrent aphtous stomatitis. In particular, said inflammatory diseases or conditions include inflammatory liver diseases like autoimmune chronic hepatitis, drug- and toxin-induced liver diseases, cirrosis, primary biliary cirrosis and primary sclerosing cholangitis.

According to a third embodiment, said inflammatory disease or condition is a central nervous system inflammatory disease or condition, such as, but not limited to multiple sclerosis, Alzheimer's disease, or ischemia-reperfusion injury associated with ischemic stroke.

According to a fourth embodiment, said inflammatory disease or condition is a pulmonary inflammatory disease or condition, such as, but not limited to asthma, chronic obstructive pulmonary disease, or adult respiratory distress syndrome.

According to a fifth embodiment, said inflammatory disease or condition is a skin inflammatory disease or condition such as, but not limited to contact dermatitis, atopic dermatitis, psoriasis, pityriasis rosea, lichen planus, or pityriasis rubra pilaris.

According to a seventh embodiment said inflammatory condition is related to tissue trauma or resulting from organ transplantations or other surgical operations.

According to an eighth embodiment, said disease related to carbohydrate metabolism is a disease such as but not limited to diabetes, atherosclerosis, vascular retinopathies, retinopathy, nephropathy, nephrotic syndrome, polyneuropathy, mononeuropathies, autonomic neuropathy, foot ulcers or joint problems.

According to a tenth embodiment said disease relating to aberrations in adipocyte differentiation or function or smooth muscle cell function is a disease such as but not limited to atherosclerosis or obesity.

According to an eleventh embodiment, the vascular disease is a disease such as but not limited to atheromatous ateriosclerosis, nonatheromateous ateriosclerosis, ischemic heart disease, peripheral aterial occlusion, thromboangiitis obliterans (Buerger's disease), or Raynaud's disease and phenomenon.

The invention will be illuminated by the following non-restrictive Experimental Section.

Experimental Section

Knock-Down of mouseVAP-1 Expression by siRNA:s In Vitro

CHO cells stably transfected with mouse VAP-1 cDNA in pCDNA3.1 expression vector were cultured to 70-90% confluency in 24 well plates in alpha-MEM medium containing 10% fetal calf serum. The siRNA oligonucleotides (the siRNA duplexes no. 1-4 as shown in FIG. 2) were transfected into cells using Lipofectamine 2000 reagent according to the manufacturer's instructions. Briefly, 1 μl Lipofectamine reagent was mixed with 50 μl Optimem medium in one tube and VAP-1 or GFP siRNA (both at 10 pmol and 50 pmol) with 50 μl Optimem medium in another tube, and the tubes were allowed to stand at room temperature for 5 min. Then the contents of the two tubes were mixed and allowed to stand for 25 min at room temperature. Meanwhile, the CHO mouse VAP-1 transfectants were rinsed twice with Optimem medium and thereafter 0.4 ml Optimem was added per well. Thereafter the mixed transfection solution was added into the wells (giving 20 nM or 100 nM final concentration of the siRNA). The cells were cultured for 4 h in a humified cell incubator at 37° C. and then the medium was replaced with the normal MEM alpha medium. The plates were then transferred back to the cell incubator for 2 days.

After the incubation the cells were detached using trypsin-edta solution and stained for immunofluorescence. In brief, the cells were incubated with a negative control mAb 3G6 or with an anti-mouse VAP-1 mAb 7-106 at 10 μg/ml for 15 min. After washings, FITC-conjugated goat anti-rat second stage reagent was added for 15 min. After washings the cells were fixed in paraformaldehyde-containing buffer and analyzed using FACSCalibur flow cytometer.

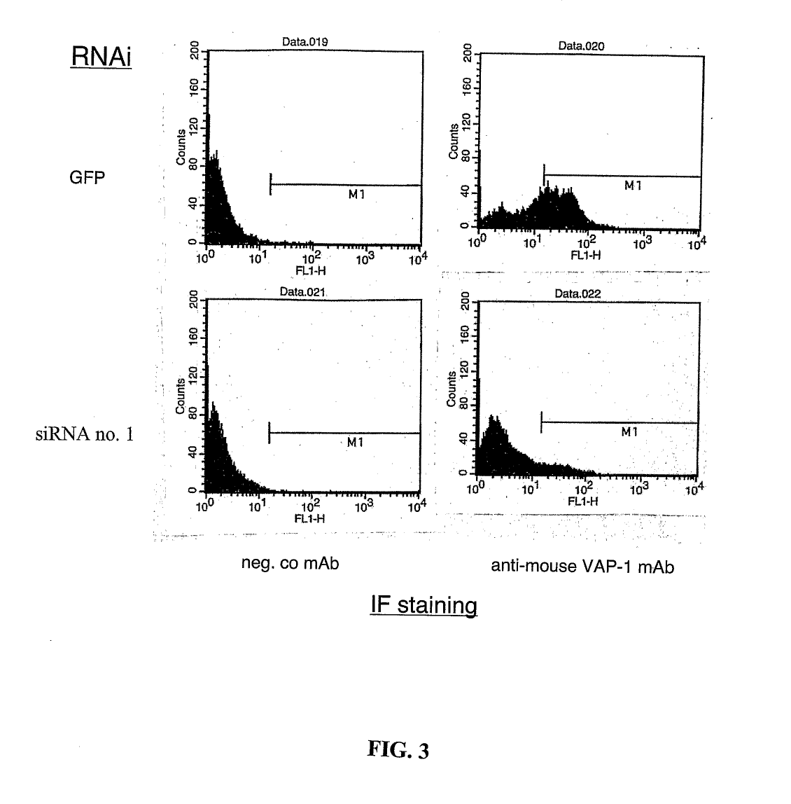

All four siRNA duplexes no. 1-4, used at 20 nM, reduced VAP-1 expression on the transfectants, whereas the control siRNA against GFP was without effect. The results with siRNA no. 1 are shown in FIG. 3.

Similar results were obtained in three independent assays with the pool and separate siRNA molecules. The siRNAs were also tested at 100 nM concentration, and the results were essentially the same.

Knock-Down of Human VAP-1 Expression by siRNA:s In Vitro

CHO cells stably transfected with human VAP-1 cDNA in pCDNA3.1 expression vector were cultured to 70-90% confluency in 24 well plates in alpha-MEM medium containing 10% fetal calf serum. The siRNA oligonucleotides (the siRNA duplexes no. I-IV as shown in FIG. 5) were transfected into cells using Lipofectamine 2000 reagent according to the manufacturer's instructions. Briefly, 1 μl Lipofectamine reagent was mixed with 50 μl. Optimem medium in one tube and VAP-1 or GFP siRNA (both at 10 pmol and 50 pmol) with 50 μl Optimem medium in another tube, and the tubes were allowed to stand at room temperature for 5 min. Then the contents of the two tubes were mixed and allowed to stand for 25 min at room temperature. Meanwhile, the CHO human VAP-1 transfectants were rinsed twice with Optimem medium and thereafter 0.4 ml Optimem was added per well. Thereafter the mixed transfection solution was added into the wells (giving 20 nM or 100 nM final concentration of the siRNA). The cells were cultured for 4 h in a humified cell incubator at 37° C. and then the medium was replaced with the normal MEM alpha medium. The plates were then transferred back to the cell incubator for 2 days.

After the incubation the cells were detached using trypsin-edta solution and stained for immunofluorescence. In brief, the cells were incubated with a negative control mAb 3G6 or with an anti-human VAP-1 mAb TK-8-14 at 10 μg/ml for 15 min. After washings, FITC-conjugated goat anti-mouse Ig second -stage reagent was added for 15 min. After washings the cells were fixed in paraformaldehyde-containing buffer and analyzed using FACSCalibur flow cytometer.

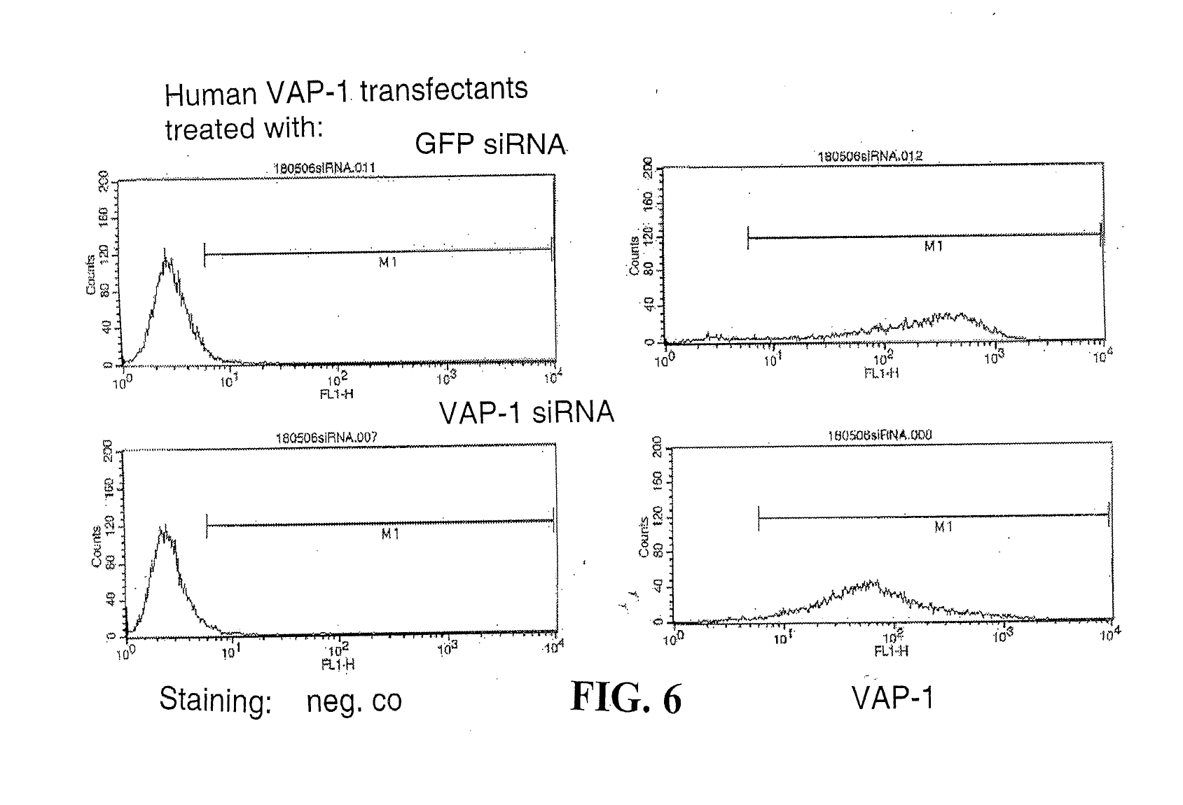

All four siRNA duplexes no. I-IV, used at 100 nM, reduced humanVAP-1 expression on the transfectants when compared to cells treated with the control siRNA against GFP. The results from one representative experiment with siRNA no. III are shown in FIG. 6, which shows a 63% reduction in the mean fluorescence intensity of VAP-1 expression after the treatment. Mean fluorescence intensity is a measure of the number of molecules per cell and thus indicates that after VAP-1 siRNA treatment the cells have lost 63% of their VAP-1 molecules. Mean reduction in the mean fluorescence intensities from two independent assays were 53% with siRNA III. siRNAs I and II both gave a mean reduction of 43% in VAP-1 expression, whereas no IV only marginally reduced VAP-1 expression by 10%.

The siRNAs were also tested at 20 nM concentration, and again no III was the most potent showing a 33% reduction in the expression. Similar results were obtained in two independent assays.

Based on these promising results for the siRNA duplexes in vivo, the inventors are currently testing their efficacy in vivo.

It has been shown that mouse VAP-1 protein is efficiently down-regulated by siRNA in vitro. Therefore, RNAi technology appears to be a promising new way to knock-down this adhesion molecule also in vivo.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. Method for prevention or treatment of a disease or disorder that benefits from the inhibition or down regulation of VAP-1
said method comprising administration of a small interfering RNA (siRNA) that down regulates the expression of vascular adhesion protein 1 (VAP-1),
said siRNA being a duplex comprising an antisense sequence of about 21 nucleotides, said antisense being complementary to a region of the VAP-1 mRNA, and a sense sequence that is complementary to a sequence of about 19 nucleotides of said antisense, wherein the antisense sequence and the sense sequence both comprise a 3′-terminal overhang of a few, typically 2 nucleotides, and wherein the 5′-terminal of the antisense is a phosphate group (P).

2. The method according to claim 1, wherein the disease or disorder is selected from the group consisting of inflammatory diseases or conditions; diseases related to carbohydrate metabolism; diseases related to aberrations in adipocyte differentiation or function or smooth muscle cell function and vascular diseases, in particular inflammatory diseases or conditions including inflammatory liver diseases like autoimmune chronic hepatitis, drug- and toxin-induced liver diseases, cirrosis, primary biliary cirrosis and primary sclerosing cholangitis.

3. The method according to claim 1, wherein the siRNA can be selected from a group consisting of siRNA no. I (SEQ ID NO 11 and 12), siRNA no. II (SEQ ID NO 13 and 14), siRNA no. III (SEQ ID NO 15 and 16) and siRNA no. IV (SEQ ID NO 17 and 18) as disclosed in FIG. 5.

4. The method according to claim 1, wherein the siRNA comprises modifications of one or more 2′-hydroxyl groups at ribose sugars, and/or modifications in one or more internucleotidic phosphodiester linkages, and/or one or more locked nucleic acid (LNA) modification between the 2′- and 4′-position of the ribose sugars.

5. A pharmaceutical composition comprising the siRNA according to claim 1 and a pharmaceutically acceptable carrier.

6. The pharmaceutical composition according to claim 5 wherein the carrier is a liposome, cholesterol, lithocholic acid, lauric acid, a cationic lipid, polyethylenimine (PEI) or its conjugates with polyethylene glycol (PEG) derivatives.

7. An expression vector comprising nucleic acid encoding the siRNA duplex or the antisense strand of said duplex according to claim 1 in a manner which allows expression of said siRNA duplex or antisense strand within a mammalian cell.

8. The expression vector according to claim 7 wherein the nucleic acid encoding said siRNA duplex or antisense strand is inserted either in a DNA sequence or in a viral vector.

9. The expression vector according to claim 8 wherein the nucleic acid encoding said siRNA or antisense strand is inserted in a viral vector and said viral vector is based on an adenovirus, an alfavirus, an adeno-associated virus or a retrovirus.

10. A pharmaceutical composition comprising an expression vector according to claim 7 and a pharmaceutically acceptable carrier.

11. The pharmaceutical composition according to claim 10 wherein said carrier is a liposome, cholesterol, lithocholic acid, lauric acid, a cationic lipid, polyethylenimine (PEI) or its conjugates with polyethylene glycol (PEG) derivatives.

12.-13. (canceled)


SEQUENCE LISTING (18 sequences)

SEQ ID NO: 1 - DNA, 4452 bp, Mus musculus
cgcggccggtcgacgaagttagtctgtggggcagacccggctactttttggcagtggatttccatacacagacgagatccctcccccttcccccaaccccagctctcccccaatcccaaccctgacccagagagcactctgatttggaaactggaggacaaagccatgtaaggtctgtggatatcactgcagccagctcagacccgcctcccaaccctagctccggcttccctgctccttcccaccctggcaaaagcagactacaaagaagctctgctattgcctagcgcctgggagaccccagccaaaaggagtccatttctggtagaagcctgtccatcaagaagcaatgacccagaagaccaccctagtgctcctggctctggctgtcatcaccatctttgctttggtttgtgtcttgctagctggcaggagcggagatgggggtggactgagccaacctcttcattgcccctctgttcttcctagtgtccagccccggacacaccctagccagagccagccgtttgcagacctgagcccagaggagctgacagctgtgatgagcttcctgaccaagcacctggggccagggctggtggatgcagcccaggctcgaccctcggacaactgtgtcttctcagtagagttgcagctgcctgccaaggctgcagccctggcccacttggacagaggggggcccccacctgtgagagaggcattggccatcatcttctttggtggacaacccaagcccaacgtgagtgagctggtggtggggcccctgcctcacccctcctacatgcgggatgtgactgtggagcgccacggcggccccctgccctattaccggcgtcctgtgttggacagagagtatcaggatatcgaggagatgatcttccacagagagctgccccaagcttctggactcctccatcactgttgcttctacaaacaccagggacagaacctgctaacaatgactacagccccccgtggtttgcaatcaggggaccgggccacctggtttggcttgtattacaatctctcaggggctgggttttaccctcaccccattggcttggagcttctgatagatcataaggccctggatcctgccctgtggaccatccagaaggtattctatcaaggccgttactatgagagtctcactcagctggaggaccagtttgaggctggcctggtgaatgtggtattggtcccaaacaatggtacaggtgggtcctggtctctaaagtcctcagtgccaccgggcccagctccccctctgcagttccatccccagggaccccggttcagtgtccaggggagccaagtgtcttcttccttgtgggctttttcttttggccttggagctttcagtggcccaaggatctttgatatccgctttcaaggggagagggtggcctatgaaatcagtgtccaggaggccatagccctctacggtggaaattccccagcatcaatgtcgacctgctacgtggacggtagctttggcattggcaaatactctacccccctgatccgaggggtagactgtccttacctcgccacctatgtggactggcacttccttctggaatctcaggcccccaagacactacgtgatgcattttgtgtgtttgaacagaaccagggcctcccactccggcggcaccactcagatttctactcccattattttgggggtgttgtggggacggtgcttgtggtcagatccgtgtctaccttgctcaattacgactacatatgggacatggtcttccaccccaatggggccatagaagtcaaattccacgccacaggctatatcagctcagctttcttcttcggtgctggtgagaagtttgggaaccgagttggggcgcacacgctgggcacggtacacacccacagcgctcacttcaaagtggatctggatgtggcagggctgaagaactgggcctgggcagaggatatggcttttgtccccacgattgtaccttggcaaccggagtaccagatgcagaggctgcaggtgactcggaagctgctggagacagaggaggaggctgccttcccactggggggcgccaccccacgctacctgtacctggccagtaaccacagcaacaagtggggtcataggcggggctaccgcatccagatactcagctttgctggaaagcccttgccccaggaaagtcccatagagaaagccttcacctgggggaggtatcacttggctgtgacccagaggaaggaggaggagcctagcagctctagcatcttcaaccagaacgacccgtggacccccactgtgaacttcaccgacttcatcagcaatgagaccattgctggagaggacttggtagcctgggtgacggctggctttttgcacatccctcatgcagaagatatccccaacacggtgactgcggggaactcagtgggcttcttcctccggccgtataacttctttgacgaggacccctcctttcattctgctgactccatctatttccgggagggccaggatgccacggcctgtgaggttaaccccttggcttgcctgtcccagactgccacctgtgcccccgaaattcctgccttctcccatgggggctttgcttacagagacaattgaactgtttctaagtatccctccctcgctcctgctcagaccatgtgctcacttccccacgccattaagtgtccccaagatggacaatctagctaagagctgggaagtagcgcaacagccgggcagtacacagagcaattcgattgaagatctggttccttctgtccccacatctttgatgtcccctctctcttctgctgccctccttgtctctccctctctgcttggagcatcctgagcccatggaaacctgatgcacagggacactgaactttgttggttgtgcctgtactgagttcctgccttgggagaatagccttgttggagcctggagtaatggctatgttttgttttgctttgaatatggctccttttccccacccccaccgcaccccctatttggctttcatttaaaagcttatgatagctttgaggactctgcaatgaggataactctctagagacccccaaagtagggtctctctgagtctctcacatccacagattcttcatctacatccctttcctacttaagcctctttcatttccattctctccctctccctccccgccccctctcctttcctccctcccccctccctcccctcttcctggagttcctgcttgctcatacatgctaggattcctactcagtccaaaagcgatgtccgtcctaagagttctctgagccctcattctcaaactgcagtctcttctcctggacccgagtctgtggaaatctcaaagatgtaatgcaaagcctcatgggagaggaccaggcttttctatctcctaacgttcgtcacatatcaggaaggagtagggcttgtccctgcggtagccctgctttgccctctttacaagaagatggaggtgctatgattatgacgaatggttattttattctatggctctgctccactaatggctctggtctctgtgcagtagcctgcaggagccagctcgggacagtgagataggatggggtctgcccaatcagaagcaggcatgcaaatgagcctgagtctgatctgccttggtgggacctgtttcagggtttctctttgctgcttgtctttgctgagacctggacaggggctggtacacacatgactcaaaacaacagatacatcattccctagcagtctggccagccccgttgttaactaatgttattactgtagttaatggaagaaaggctggaaagatgtgtgtgcactgttcttgcaaggggcccaaattccgttcccagcatccttattaaggtgctcacaacaatctataactctatctccaggagagccggtgccctcttcaggggcacctaaacttaatgcacataccttcacacagacacatacatataaatacaaaataaaagaactcagcctggcacggtgacacatgactttaatcccagcattggaagtcagaggtagacagatctctgagcatgagtccagcctggtctacagagtgagttccaggacagccatagctatacagagaaaccctgtcttgcaaaacaaaaacaaacaaaccaaaagagagttctttaaagacaaaagaaaagctaaaaatgagtaggcgataagtgtacatttgtttatttctgctgcattccttgagaactgagtaagaccagttacctctgaggaggaagacagctgagagggaatcaggttctgcctgcccttctgaacatcatggattggaaccatgtgatcatattgccccgtcaaaataaataagtgggctataaaaaaaaaaaaaaaaaaaaaaaaaaaaaaaaaaaaaaaaaaaaaaaa

SEQ ID NO: 2 - RNA, 21 nt, Artificial
  siRNA No. 1 sense
gacagaaccugcuaacaauuu

SEQ ID NO: 3 - RNA, 21 nt, Artificial
  siRNA No. 1 antisense
auuguuagcagguucugucuu

SEQ ID NO: 4 - RNA, 21 nt, Artificial
  siRNA No. 2 sense
gcucaauuacgacuacauauu

SEQ ID NO: 5 - RNA, 21 nt, Artificial
  siRNA No. 2 antisense
uauguagucguaauugagcuu

SEQ ID NO: 6 - RNA, 21 nt, Artificial
  siRNA No. 3 sense
ccauagaagucaaauuccauu

SEQ ID NO: 7 - RNA, 21 nt, Artificial
  siRNA No. 3 antisense
uggaauuugacuucuaugguu

SEQ ID NO: 8 - RNA, 21 nt, Artificial
  siRNA No. 4 sense
gccacggccugugagguuauu

SEQ ID NO: 9 - RNA, 21 nt, Artificial
  siRNA No. 4 antisense
uaaccucacaggccguggcuu

SEQ ID NO: 10 - DNA, 4040 bp, Homo sapiens
gtccttcccacccttagtcccaggcatctgactaccgggaacctcagccagagtccgggagccccccaccccgtccaggagccaacagagcccccgtcttgctggcgtgagaatacattgctctcctttggttgaatcagctgtccctcttcgtgggaaaatgaaccagaagacaatcctcgtgctcctcattctggccgtcatcaccatctttgccttggtttgtgtcctgctggtgggcaggggtggagatgggggtgaacccagccagcttccccattgcccctctgtatctcccagtgcccagccttggacacaccctggccagagccagctgtttgcagacctgagccgagaggagctgacggctgtgatgcgctttctgacccagcggctggggccagggctggtggatgcagcccaggcccggccctcggacaactgtgtcttctcagtggagttgcagctgcctcccaaggctgcagccctggctcacttggacagggggagccccccacctgcccgggaggcactggccatcgtcttctttggcaggcaaccccagcccaacgtgagtgagctggtggtggggccactgcctcacccctcctacatgcgggacgtgactgtggagcgtcatggaggccccctgccctatcaccgacgccccgtgctgttccaagagtacctggacatagaccagatgatcttcaacagagagctgccccaggcttctgggcttctccaccactgttgcttctacaagcaccggggacggaacctggtgacaatgaccacggctccccgtggtctgcaatcaggggaccgggccacctggtttggcctctactacaacatctcgggcgctgggttcttcctgcaccacgtgggcttggagctgctagtgaaccacaaggcccttgaccctgcccgctggactatccagaaggtgttctatcaaggccgctactacgacagcctggcccagctggaggcccagtttgaggccggcctggtgaatgtggtgctgatcccagacaatggcacaggtgggtcctggtccctgaagtcccctgtgcccccgggtccagctccccctctacagttctatccccaaggcccccgcttcagtgtccagggaagtcgagtggcctcctcactgtggactttctcctttggcctcggagcattcagtggcccaaggatctttgacgttcgcttccaaggagaaagactagtttatgagataagcctccaagaggccttggccatctatggtggaaattccccagcagcaatgacgacccgctatgtggatggaggctttggcatgggcaagtacaccacgcccctgacccgtggggtggactgcccctacttggccacctacgtggactggcacttccttttggagtcccaggcccccaagacaatacgtgatgccttttgtgtgtttgaacagaaccagggcctccccctgcggcgacaccactcagatctctactcgcactactttgggggtcttgcggaaacggtgctggtcgtcagatctatgtccaccttgctcaactatgactatgtgtgggatacggtcttccaccccagtggggccatagaaatacgattctatgccacgggctacatcagctcggcattcctctttggtgctactgggaagtacgggaaccaagtgtcagagcacaccctgggcacggtccacacccacagcgcccacttcaaggtggatctggatgtagcaggactggagaactgggtctgggccgaggatatggtctttgtccccatggctgtgccctggagccctgagcaccagctgcagaggctgcaggtgacccggaagctgctggagatggaggagcaggccgccttcctcgtgggaagcgccacccctcgctacctgtacctggccagcaaccacagcaacaagtggggtcacccccggggctaccgcatccagatgctcagctttgctggagagccgctgccccaaaacagctccatggcgagaggcttcagctgggagaggtaccagctggctgtgacccagcggaaggaggaggagcccagtagcagcagcgttttcaatcagaatgacccttgggcccccactgtggatttcagtgacttcatcaacaatgagaccattgctggaaaggatttggtggcctgggtgacagctggttttctgcatatcccacatgcagaggacattcctaacacagtgactgtggggaacggcgtgggcttcttcctccgaccctataacttctttgacgaagacccctccttctactctgccgactccatctacttccgaggggaccaggatgctggggcctgcgaggtcaaccccctagcttgcctgccccaggctgctgcctgtgcccccgacctccctgccttctcccacgggggcttctctcacaactaggcggtcctgggatggggcatgtggccaagggctccagggccagggtgtgagggatggggagcagctgggcactgggccggcagcctggttccctctttcctgtgccaggactctctttcttccactaccctccctcgcatccgcctctgagccaggagcctcctgaccctgtgatgcctgacacaggggacactgaaccttgttgatgccagctgtactgagttctcatccacagaggccaggcatggcccagcctggagccgtggccgagggcttccctagatggttccctttgttgctgtctggctttcccgaatctttttaggccacctccaaggactctaaaagggggctattccctggagaccccagagtagggttgccagtcctgcaagtccatagctgagctggaaaggatgcttctgctcacattccctctcatccaggtcctttccttctcgtcttcctctctctcacctacttcctcctcctcctcctgttcctgccttctcttctatcctgcaatttctcccgaatcctgaggggatatccctatgtcccagcccctggtactcccccagccctcagttttcagtcaagttccgtctcctctccagccctatggaagtctcaaggtcacgggacccctaatcagagtggccaatccctgtgtgtcgttcccttgtgtctgttgcttattgggagtaggagttgctcctacccctgtcctggggctgggtgtgtttcaggacagctgcttctgtgcatttgtgtctgcctgcctcatgctctctatagaggaggatggtcatcgtgacagcagcagctcaagttagcatttcaagtgatttgggggtgcaatgataatgaagaatggccattttgtaccagggctctgtattctgcaacagcctgtttgggaggctggagtggaaacaaagggtgggcatcaaagatgagaagccaaagcccctacaactccagccacccagccaggaggggctgtccaatcacattcaggcatgcgaatgagctgggccctgggtgaggtgggggtctggcctagtggggaggggcctggcctgggtggggcagggcctggcctggtccaggcttgggctccattcccatcactgctgtccctcctgaggtctggattggggatggggacaaagaaatagcaagagatgagaaacaacagaaacttttttctctaaaggactggttaaatcaattctgatacagccttacaatacaatagtatgcagctaaaaaataattgtatgtctttatatactaatatgtaataatcttcaggtgaaaaaggcaagccacagaaatgtgtatagcgcacttcccatttgtgtttcagaaaggagtagaatataaacacataattgcttatgtatgcctattcagaataaatgggtaacactgattacttttgggaggggaaccagtaggttgaggacaggagagggaagggtcttaacacttacacccttttgtacattttgaattttgaaccatgtgactgtattacctattcaaaataaacaataaatgggcccaaaaaaaaaaaaaaaaa

SEQ ID NO: 11 - RNA, 21 nt, Artificial
  siRNA No. I sense strand
uuaggguccuggcggaucaac

SEQ ID NO: 12 - RNA, 21 nt, Artificial
  siRNA No. I antisense strand
ucccaggaccgccuaguuguu

SEQ ID NO: 13 - RNA, 21 nt, Artificial
  siRNA No. II sense strand
uuaguauuugaucagaaagag

SEQ ID NO: 14 - RNA, 21 nt, Artificial
  siRNA No. II antisense strand
ucauaaacuagucuuucucuu

SEQ ID NO: 15 - RNA, 21 nt, Artificial
  siRNA No. III sense strand
uuaguaucaacucguuccacc

SEQ ID NO: 16 - RNA, 21 nt, Artificial
  siRNA No. III antisense strand
ucauaguugagcaaggugguu

SEQ ID NO: 17 - RNA, 21 nt, Artificial
  siRNA No. IV sense strand
uuuaggaaaggucguuaccag

SEQ ID NO: 18 - RNA, 21 nt, Artificial
  siRNA No. IV antisense strand
auccuuuccagcaauggucuu